(19) United States
(12) Patent Application Publication
(10) Pub. No.: US 2004/0105884 A1
(43) Pub. Date: Jun. 3, 2004

(54) Pharmaceutical dosage form comprising a sulfite compound

(76) Inventors: Ping Gao, Portage, MI (US); Juliane M. Bauer, Portage, MI (US); Gary D. Ewing, Kalamazoo, MI (US); David C. Sperry, Kalamazoo, MI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/632,737

(22) Filed: Jul. 31, 2003

Related U.S. Application Data
(63) Continuation-in-part of application No. 10/119,129, filed on Apr. 9, 2002.
(60) Provisional application No. 60/284,381, filed on Apr. 17, 2001.
(60) Provisional application No. 60/326,952, filed on Oct. 4, 2001.
(60) Provisional application No. 60/399,862, filed on Jul. 31, 2002.
(60) Provisional application No. 60/399,776, filed on Jul. 31, 2002.
(60) Provisional application No. 60/399,863, filed on Jul. 31, 2002.
(60) Provisional application No. 60/399,808, filed on Jul. 31, 2002.

(51) Int. Cl. A61K 31/415; A61K 9/64; A61K 31/355; A61K 33/04

(52) U.S. Cl. 424/456; 424/703; 514/406; 514/473; 514/458; 514/474

(57) ABSTRACT

The present invention provides a pharmaceutical dosage form comprising a fill material sealed in a gelatin capsule; the fill material comprises (a) a selective COX-2 inhibitory drug of low water solubility, and (b) a sulfite compound in an amount sufficient to inhibit cross-linking of gelatin in said gelatin capsule upon storage of the dosage form in a closed container maintained at 40° C. and 75% relative humidity for a period of 6 months.

This application is a continuation-in-part of U.S. application Ser. No. 10/119,129 filed on 9 Apr. 2002, which claims priority of U.S. provisional application Serial No. 60/284,381 filed on 17 Apr. 2001 and U.S. provisional application Serial No. 60/326,952 filed on 4 Oct. 2001. This application also claims priority of U.S. provisional application Serial No. 60/399,862 filed on 31 Jul. 2002, U.S. provisional application Serial No. 60/399,776 filed on 31 Jul. 2002, U.S. provisional application Serial No. 60/399,863 filed on 31 Jul. 2002, and U.S. provisional application Serial No. 60/399,808 filed on 31 Jul. 2002.

### FIELD OF THE INVENTION

The present invention relates to gelatin capsules filled with a fill material comprising a selective COX-2 inhibitory drug of low water solubility and a sulfite compound and to reduced gelatin cross-linking thereof. BACKGROUND OF THE INVENTION

Gelatin, a mixture of water-soluble proteins derived from collagen by hydrolysis, is widely used in the pharmaceutical and food industries, among others. One major application of gelatin is in preparation of both hard and soft gelatin capsules. Such capsules are desirable for, inter alia, their versatility (they may contain drug formulations in solid, semi-solid, or liquid form) and for their rapid dissolution characteristics. Unfortunately, drug dosage forms containing gelatin in an outer layer (e.g. liquid or powder filled into a gelatin capsule) can exhibit a drop in dissolution rate over time. This drop in dissolution rate can lead to undesirable and unacceptable alterations in in vitro dissolution profile and in bioavailability, particularly for drugs of low water solubility or drugs whose absorption is dissolution-rate limited. Such changes in dissolution profile are thought to result from cross-linking of gelatin occurring in gelatin capsule shells.

Singh et al., Alteration in Dissolution Characteristics of Gelatin-Containing Formulations, Pharmaceutical Technology, April 2002, hereby incorporated by reference herein but not admitted to be prior art, describes reports suggesting that several agents including glycerine, glycine, and hydroxylamine hydrochloride, when incorporated into fill contents of gelatin capsules, can limit gelatin cross-linking. Unfortunately, existing methods directed at the problem of gelatin cross-linking in capsule shells are less than satisfactory, particularly in situations where longer shelf life and stability through real life storage, shipping and handling conditions are desired, pursuit of adequate solutions to the problem of gelatin capsule cross-linking is therefore desired.

If a pharmaceutical dosage form comprising a fill material in a gelatin capsule could be prepared which are capable of providing a predictable and stable drug dissolution rate, even after storage under stressed conditions, a significant advance in the oral delivery of drugs, particularly drugs of low water solubility or drugs whose absorption is dissolution-rate limited, would result.

### SUMMARY OF THE INVENTION

There is now provided in the present invention a pharmaceutical dosage form comprising a fill material sealed in a gelatin capsule shell, the fill material comprising (a) a selective COX-2 inhibitory drug of low water solubility, and (b) a sulfite compound. in an amount sufficient to inhibit cross-linking and/or pellicle formation of the gelatin capsule shell upon storage.

Desirably, the sulfite compound in the dosage form is present at a concentration sufficient to (a) inhibit cross-linking of the gelatin and/or pellicle formation in the capsule shell and/or (b) provide a dosage form with a stable dissolution profile.

The dosage form of the present invention is especially useful for liquid fill materials and for soft gelatin capsules.

The term “pellicle” herein refers to a relatively water-insoluble membrane formed in a gelatin capsule shell. Such a membrane tends to be thin, tough, and rubbery. It is now understood that one mechanism underlying pellicle formation is gelatin cross-linking. Gelatin cross-linking and pellicle formation result in reduced dissolution rates. Accordingly, quantification of dissolution rate of a first capsule within a reasonably short time after capsule preparation and of a second capsule after storage under stressed conditions (e.g., four weeks at 40° C. and 85% relative humidity in a closed container) as described herein provides one means of assessing pellicle formation and/or gelatin cross-linking. The term “within a reasonably short time after capsule formation” means within a period such that substantial cross-linking and/or pellicle formation is unlikely to have yet occurred, for example within one week, dependent upon storage conditions during that period.

The term “pellicle-resistant” herein means that a gelatin capsule so described has a reduced tendency to form, or exhibits slowed, delayed or reduced formation of a pellicle upon storage under stressed conditions. Similarly, “inhibition of cross-linking” (or “inhibition of pellicle formation”) herein means a slowed, delayed or reduced formation of gelatin cross-links (or pellicle formation) in a capsule by comparison with an otherwise similar capsule lacking only the amine agent as provided herein.

Pharmaceutical dosage forms according to the present invention have been found to exhibit an unexpected and surprisingly substantial reduction in cross-linking of gelatin in the gelatin capsule shell and reduction in pellicle formation. As a result, such dosage forms are capable of consistently meeting desired in vitro dissolution criteria, even after storage under stressed conditions. This invention represents a significant improvement over conventional dosage forms.

### BRIEF DESCRIPTION OF THE DRAWINGS

FIG. 1 is a graph showing Tier I dissolution rate of Formulation 30 following storage at 25° C. as described in Example 3.

FIG. 2 is a graph showing Tier I dissolution rate of Formulation 30 following storage at 40° C. as described in Example 3.

FIG. 3 is a graph showing Tier II dissolution rate of Formulation 30 following storage at 25° C. as described in Example 3.

FIG. 4 is a graph showing Tier II dissolution rate of Formulation 30 following storage at 40° C. as described in Example 3.

FIG. 5 is a graph showing Tier I dissolution rate of Formulation 19 following storage at 25° C. as described in Example 3.

FIG. 6 is a graph showing Tier I dissolution rate of Formulation 19 following storage at 40° C. as described in Example 3.

FIG. 7 is a graph showing Tier II dissolution rate of Formulation 19 following storage at 40° C. as described in Example 3.

### DETAILED DESCRIPTION OF THE INVENTION

In one embodiment, the present invention provides a dosage form comprising a fill material sealed in a gelatin capsule shell, the fill material comprising (a) a selective cyclooxygenase-2 inhibitory drug of low water solubility and (b) at least one pharmaceutically acceptable sulfite compound in an amount sufficient to inhibit cross-linking and/or pellicle formation in the gelatin capsule shell upon storage.

Gelatin Cross-Linking, Pellicle Formation, and Drug Dissolution.

Without being bound by theory, the inventors' believe that gelatin cross-linking can result from a process by which amino acid residues of gelatin covalently bond to form an insoluble material. The process can be the result of low levels of aldehydes coming into contact with the gelatin. Cross-linking of a gelatin capsule can impact product performance by delaying the release of the formulation (containing the active compound) from the capsule shell. The delay in release can, in turn, affect the rate of absorption of the compound into the blood stream and clinical onset of action. While ‘mild’ cross-linking does not necessarily have a significant impact on release of the formulation from the dosage form, ‘severe’ cross-linking can have a significant impact. When cross-linking is severe, it can lead to a delay of release of formulation from the dosage form in humans, potential bioequivalence problems, and a potential delay in clinical onset of action.

Dosage forms of the present invention exhibit decreased gelatin cross-linking (and pellicle formation) and, therefore, when placed in an in vitro dissolution assay, are capable of advantageously exhibiting less dissolution rate change during storage under stressed conditions than conventional dosage forms. Dosage forms according to the present invention also exhibit more uniform inter-dosage form drug dissolution rate than standard dosage forms.

In one embodiment of the present invention wherein the fill material further comprises at least one substance that promotes cross-linking of gelatin when in contact therewith (the substance being the drug itself or an excipient substance, and the substance acting independently or in combination with one or more other substances to promote said cross-linking); upon (a) immediately testing a first dosage form in a first in vitro dissolution assay; (b) storing a second dosage form which is identical to the first dosage form in a closed container maintained at 40° C. and 75% relative humidity for a period of four weeks and, after said storage; (c) testing the second dosage form in a second in vitro dissolution assay which is identical to the first in vitro dissolution assay; the amount of drug dissolved at 45 minutes in the second dissolution assay is within ±15 percent and preferably within ±10 percent of the amount of drug dissolved at 45 minutes in the first dissolution assay.

Because gelatin cross-linking may lead to delayed dissolution, storage time-dependent delays in dissolution profile may be a good indicator of gelatin cross-linking during such storage. There are a number of in vitro dissolution assays suitable for determining dissolution profile. Indeed, one skilled in the art is able to design additional assays or modifications thereof. Two dissolution-type test methods were developed and set forth herein and designated the “Tier I” and “Tier II ” tests.

In the Tier I test, a dosage form is placed in a USP apparatus II with a rotating paddle with a paddle speed of 50 rpm in 900 mL of 0.01N HCl +1% Tween 80. Samples are typically withdrawn at 15, 30, 45, 60 and 90 minutes and assayed for drug content by HPLC.

The Tier II test employs the addition of the enzyme pepsin to the medium. Pepsin in the human stomach digests cross-linked gelatin. The appropriate amount of pepsin added to the media (750,000 units/L) was determined and reported in Collaborative Development of Two-Tier Dissolution Testing for Gelatin Capsules and Gelatin-Coated Tablets using Enzyme-Containing Media, Stimuli to the Revision Process, Pharmacopeial Forum, Vol. 25, No. 5, September-October 1998. The Tier II drug release test designed in this way is expected to produce a drug release profile that is a reasonable approximation of the drug release profile in humans.

An ‘initial’ drug release profile is determined for each dosage form within a reasonably short time after preparation (i.e. before the dosage form is exposed to conditions that might result in gelatin cross-linking, such as temperature or relative humidity). A subsequent profile is determined for samples pulled at subsequent time points. A change from initial to subsequent Tier I profile (i.e. a delay in dissolution) is presumptively attributed to gelatin cross-linking. When such a change is reduced in the Tier II assay (containing pepsin), this reduction is deemed further evidence of gelatin cross-linking upon storage.

Fill Material

i. Selective Cyclooxygenase-2 Inhibitory Drug

Dosage forms of the invention comprise a selective cyclooxygenase-2 inhibitory drug, also referred to herein as a selective COX-2 inhibitory drug. Preferably, the COX-2 inhibitory drug is a drug of low water solubility (e.g. having a room temperature solubility in water of not more than about 10 mg/ml and more preferably not more than about 1 mg/ml). A preferred selective COX-2 inhibitory drug useful herein, or to which a salt or prodrug useful herein is converted in vivo, is a compound of formula (I)

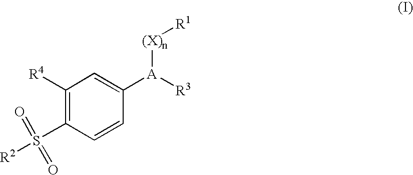

wherein:

A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;

X is O, S or CH₂;

n is 0 or 1;

R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is methyl, amino or aminocarbonylalkyl;

R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and

R⁴ is selected from hydrido and halo.

Dosage forms of the invention are especially useful for selective COX-2 inhibitory drugs having the formula (II):

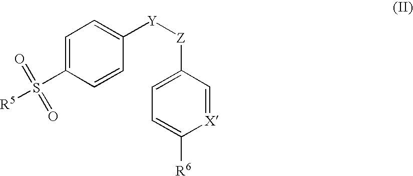

where R⁵ is a methyl or amino group, R⁶ is hydrogen or a C₁₋₄ alkyl or alkoxy group, X′ is N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, or an isomer, tautomer, pharmaceutically-acceptable salt or prodrug thereof. Preferred such five- to six-membered rings are cyclopentenone, furanone, methylpyrazole, isoxazole and pyridine rings substituted at no more than one position.

Illustratively, dosage forms of the invention are suitable for celecoxib, deracoxib, valdecoxib, rofecoxib, etoricoxib, 2-(3,5-difluorophenyl)-3-[4-(methylsulfonyl)phenyl]-2-cyclopenten-1-one, 2-(3,4-difluorophenyl)-4-(3-hydroxy-3-methyl-1-butoxy)-5-[4-(methylsulfonyl)phenyl]-3-(2H)-pyridazinone, pharmaceutically acceptable salts and prodrugs thereof. A particularly useful prodrug of valdecoxib for use in dosage forms of the invention is parecoxib, more particularly parecoxib sodium.

Dosage forms of the invention are also useful for compounds having the formula (III):

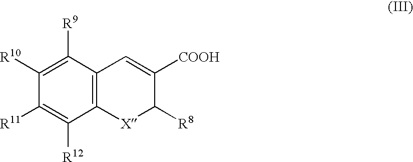

where X″ is O, S or N-lower alkyl; R⁸ is lower haloalkyl; R⁹ is hydrogen or halogen; R¹⁰ is hydrogen, halogen, lower alkyl, lower alkoxy or haloalkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen-containing heterocyclosulfonyl; and R¹¹ and R¹² are independently hydrogen, halogen, lower alkyl, lower alkoxy, or aryl; and for pharmaceutically acceptable salts thereof.

An especially useful compound of formula (III) is (S)-6,8-dichloro-2-(trifluoromethyl)-2H-1-benzopyran-3-carboxylic acid, particularly in the form of a water-soluble salt thereof, for example the sodium salt.

Where the drug is celecoxib, the dosage form typically comprises celecoxib in a therapeutically and/or prophylactically effective total amount of about 10 mg to about 1000 mg per dose unit. Where the drug is a selective COX-2 inhibitory drug other than celecoxib, the amount of the drug per dose unit is therapeutically equivalent to about 10 mg to about 1000 mg of celecoxib.

It will be understood that a therapeutically and/or prophylactically effective amount of a drug for a subject is dependent inter alia on the body weight of the subject. A “subject” herein to which a therapeutic agent or composition thereof can be administered includes a human patient of either sex and of any age, and also includes any nonhuman animal, particularly a domestic or companion animal, illustratively a cat, dog or horse.

Where the subject is a child or a small animal (e.g., a dog), for example, an amount of celecoxib relatively low in the preferred range of about 10 mg to about 1000 mg is likely to be consistent with therapeutic effectiveness. Where the subject is an adult human or a large animal (e.g., a horse), therapeutic effectiveness is likely to require dose units containing a relatively greater amount of celecoxib. For an adult human, a therapeutically effective amount of celecoxib per dose unit in a dosage form of the present invention is typically about 10 mg to about 400 mg. Especially preferred amounts of celecoxib per dose unit are about 100 mg to about 200 mg, for example about 100 mg or about 200 mg.

For other selective COX-2 inhibitory drugs, an amount of the drug per dose unit can be in a range known to be therapeutically effective for such drugs. Preferably, the amount per dose unit is in a range providing therapeutic equivalence to celecoxib in the dose ranges indicated immediately above.

ii. Sulfite Compound

Dosage forms of the invention comprise at least one pharmaceutically acceptable sulfite compound. Illustrative pharmaceutically acceptable sulfite compounds include sodium metabisulfite, sodium bisulfite, and sodium thiosulfate (sodium hyposulfite). One or more sulfite compounds are present in a composition of the invention in an amount of not more than about 10%, for example about 0.01% to about 10%, preferably about 0.1% to about 5%, and more preferably about 0.1% to about 2% of the dosage form on a dry weight basis. It should be understood that “on a dry weight basis” means total weight excepting water weight. Similar meaning is conferred by the term “by weight” unless context indicates otherwise. The sulfite compound can additionally, if desired, be present in the gelatin capsule shell. Preferably, however, at least about 50%, more preferably at least about 55%, even more preferably at least about 60%, and still more preferably at least about 65% of any sulfite compound(s) present in a dosage form of the invention are present in the fill material.

ii. Amine Agents.

Fill material of the present invention optionally further comprises an amine agent comprising at least one pharmaceutically acceptable primary or secondary amine compound. Any pharmaceutically acceptable primary or secondary amine compound can, if desired, be used in a dosage form of the invention. The term primary or secondary amine compound herein includes those primary and secondary amines which are pharmaceutically acceptable excipients. Preferably, amine agents of the present invention are compounds that are not therapeutically or nutritionally active. Non-limiting examples of suitable primary amine compounds include tromethamine (Tris), ethanolamine, ethylenediamine, diethylamine, ethylene N-methyl-D-glucamine, and amino acids such as L-arginine, L-lysine, and guanidine. Non-limiting examples of suitable secondary amine compounds include diethanolamine, benethamine (i.e., N-phenymethyl)benezeneethanamine), benzathine (i.e., N,N-di benzylethylenedi amine), piperazine, hydrabamine (i.e., N,N-bis(dehydroabietyl)ethylenediamine), and imidazole.

If present, the primary or secondary amine compound is present in a dosage form of the invention in a total amount of not more than about 10%, preferably not more than about 7%, and more preferably not more than about 5% of the dosage form on a dry weight basis, for example about 0.1% to about 4%. In one embodiment, at least about 50%, preferably at least about 55%, more preferably at least about 60%, and still more preferably at least about 65% of any primary or secondary amine compound present in a dosage form of the invention is present in the fill material.

iii. Other Excipients

Optionally, a fill material according to the invention can comprise any additional pharmaceutically acceptable excipients. Such excipients can include, by way of illustration and not limitation, diluents, disintegrants, dispersants, binding agents, adhesives, wetting agents, lubricants, glidants, crystallization inhibitors, stabilizers, antioxidants, substances added to mask or counteract a disagreeable taste or odor, flavors, dyes, fragrances, preservatives, and substances added to improve appearance of the dosage form.

Such optional additional components should be physically and chemically compatible with the other ingredients of the fill material and should not be deleterious to the recipient. Importantly, some of the above-listed classes of excipients overlap each other.

Fill material of the present invention optionally further comprises at least one pharmaceutically acceptable free radical-scavenging antioxidant. A free radical-scavenging antioxidant is to be contrasted with a “non-free radical-scavenging antioxidant”, i.e., an antioxidant that does not possess free radical-scavenging properties. Non-limiting illustrative examples of suitable free radical-scavenging antioxidants include α-tocopherol (vitamin E), ascorbic acid (vitamin C) and salts thereof including sodium ascorbate and ascorbic acid palmitate, butylated hydroxyanisole (BHA), butylated hydroxytoluene (BHT), fumaric acid and salts thereof, hypophosphorous acid, malic acid, alkyl gallates, for example propyl gallate, octyl gallate and lauryl gallate, sodium sulfite, sodium bisulfite and sodium metabisulfite. Preferred free radical-scavenging antioxidants are alkyl gallates, vitamin E, BHA and BHT. More preferably the at least one free radical-scavenging antioxidant is propyl gallate.

One or more free radical-scavenging antioxidants are optionally present in dosage forms of the invention in a total amount effective to substantially reduce formation of an addition compound, typically in a total amount of about 0.01% to about 5%, preferably about 0.01% to about 2.5%, and more preferably about 0.01% to about 1% of the dosage form on a dry weight basis.

Fill material according to the invention optionally comprises one or more pharmaceutically acceptable sweeteners. Non-limiting examples of suitable sweeteners include mannitol, propylene glycol, sodium saccharin, acesulfame K, neotame and aspartame. Alternatively or in addition, a viscous sweetener such as sorbitol solution, syrup (sucrose solution) or high-fructose corn syrup can be used and, in addition to sweetening effects, can also be useful to increase viscosity and to retard sedimentation.

Fill material of the invention optionally comprises one or more pharmaceutically acceptable preservatives other than free radical-scavenging antioxidants. Non-limiting examples of suitable preservatives include benzalkonium chloride, benzethonium chloride, benzyl alcohol, chlorobutanol, phenol, phenylethyl alcohol, phenylmercuric nitrate, thimerosal, etc.

Fill material of the invention optionally comprises one or more pharmaceutically acceptable wetting agents. Surfactants, hydrophilic polymers and certain clays can be useful as wetting agents to aid in dissolution and/or dispersion of a hydrophobic drug such as celecoxib. Non-limiting examples of suitable surfactants include benzalkonium chloride, benzethonium chloride, cetylpyridinium chloride, dioctyl sodium sulfosuccinate, nonoxynol 9, nonoxynol 10, octoxynol 9, poloxamers, polyoxyethylene (8) caprylic/capric mono- and diglycerides (e.g., Labrasol™ of Gattefossé), polyoxyethylene (35) castor oil, polyoxyethylene (20) cetostearyl ether, polyoxyethylene (40) hydrogenated castor oil, polyoxyethylene (10) oleyl ether, polyoxyethylene (40) stearate, polysorbate 20, polysorbate 40, polysorbate 60, polysorbate 80 (e.g., Tween™ 80 of ICI), propylene glycol laurate (e.g., Lauroglycol™ of Gattefossé), sodium lauryl sulfate, sorbitan monolaurate, sorbitan monooleate, sorbitan monopalmitate, sorbitan monostearate, tyloxapol, and mixtures thereof.

Additionally, dosage forms of the invention optionally comprise one or more pharmaceutically acceptable buffering agents, flavoring agents, colorants, stabilizers and/or thickeners. Buffers can be used to control pH of a formulation and can thereby modulate drug solubility. Flavoring agents can enhance patient compliance by making the dosage form more palatable, and colorants can provide a product with a more aesthetic and/or distinctive appearance. Non-limiting examples of suitable colorants include D&C Red No. 33, FD&C Red No. 3, FD&C Red No. 40, D&C Yellow No. 10, and C Yellow No. 6.

Liquid Fill Material

In a preferred embodiment, fill material comprising the selective COX-2 inhibitory drug is in the form of a liquid. More preferably, the fill material is self-emulsifying upon contact with simulate gastric fluid.

Solvents

Fill material according to this embodiment comprises at least one solvent which is preferably suitable for dissolving the drug and/or any additional ingredients or excipients present therein.

### i. Glycols and Glycol Ethers

A preferred solvent is a glycol or glycol ether. Suitable glycol ethers include those conforming to formula (X):

R¹—O—((CH₂)ₘO)ₙ—R² (X)

wherein R¹ and R² are independently hydrogen or C₁₋₆ alkyl, C₁₋₆ alkenyl, phenyl or benzyl groups, but no more than one of R¹ and R² is hydrogen; m is an integer of 2 to about 5; and n is an integer of 1 to about 20. It is preferred that one of R¹ and R² is a C₁₋₄ alkyl group and the other is hydrogen or a C₁₋₄ alkyl group; more preferably at least one of R¹ and R² is a methyl or ethyl group. It is preferred that m is 2. It is preferred that n is an integer of 1 to about 4, more preferably 2.

Glycol ethers used as solvents in fill material typically have a molecular weight of about 75 to about 1000, preferably about 75 to about 500, and more preferably about 100 to about 300. Importantly, the glycol ethers used in fill material of this embodiment must be pharmaceutically acceptable and must meet all other conditions prescribed herein.

Non-limiting examples of glycol ethers that may be used in fill material of this embodiment include ethylene glycol monomethyl ether, ethylene glycol dimethyl ether, ethylene glycol monoethyl ether, ethylene glycol diethyl ether, ethylene glycol monobutyl ether, ethylene glycol dibutyl ether, ethylene glycol monophenyl ether, ethylene glycol monobenzyl ether, ethylene glycol butylphenyl ether, ethylene glycol terpinyl ether, diethylene glycol monomethyl ether, diethylene glycol dimethyl ether, diethylene glycol monoethyl ether, diethylene glycol diethyl ether, diethylene glycol divinyl ether, ethylene glycol monobutyl ether, diethylene glycol dibutyl ether, diethylene glycol monoisobutyl ether, triethylene glycol dimethyl ether, triethylene glycol monoethyl ether, triethylene glycol monobutyl ether, tetraethylene glycol dimethyl ether, and mixtures thereof. See for example Flick (1998): Industrial Solvents Handbook, 5th ed., Noyes Data Corporation, Westwood, N.J. A particularly suitable glycol ether solvent is diethylene glycol monoethyl ether, sometimes referred to in the art as DGME or ethoxydiglycol. It is available for example under the trademark Transcutol™ of Gattefossé Corporation.

Glycols suitable as solvents in fill material include propylene glycol, 1,3-butanediol and polyethylene glycols. A presently preferred solvent is polyethylene glycol (PEG).

Any pharmaceutically acceptable PEG can be used. Preferably, the PEG has an average molecular weight of about 100 to about 10,000, and more preferably about 100 to about 1,000. Still more preferably, the PEG is of liquid grade. Non-limiting examples of PEGs that can be used in solvent liquids of this invention include PEG-200, PEG-350, PEG-400, PEG-540 and PEG-600. See for example Flick (1998), op. cit., p. 392. A presently preferred PEG has an average molecular weight of about 375 to about 450, as exemplified by PEG-400.

PEGs such as PEG-400 have many desirable properties as solvents for poorly water-soluble drugs. In the case of celecoxib, for example, the drug can be dissolved or solubilized at a very high concentration in PEG-400, enabling formulation of a therapeutically effective dose in a very small volume of solvent liquid. This is especially important where the resulting solution is to be encapsulated, as capsules of a size convenient for swallowing can be prepared containing a therapeutically effective dose even of a drug such as celecoxib having a relatively high dose requirement for efficacy. Importantly, ethanol, water, and other excipients identified as co-solvents hereinbelow or elsewhere can, if desired, be used as solvents in a fill material of the invention. Typically, one or more solvents will be present in a fill material in a total amount of about 5% to about 95%, preferably about 10% to about 90% and more preferably about 15% to about 85% of the dosage form on a dry weight basis.

Co-Solvents.

A fill material of this embodiment optionally comprises one or more pharmaceutically acceptable co-solvents. Non-limiting examples of suitable co-solvents include additional glycols, alcohols, for example ethanol and n-butanol; oleic and linoleic acid triglycerides, for example soybean oil; caprylic/capric triglycerides, for example Miglyol™ 812 of Huls; caprylic/capric mono- and diglycerides, for example Capmul# MCM of Abitec; polyoxyethylene caprylic/capric glycerides such as polyoxyethylene (8) caprylic/capric mono- and diglycerides, for example Labrasol# of Gattefosse; propylene glycol fatty acid esters, for example propylene glycol laurate; polyoxyethylene (35) castor oil, for example Cremophor™ EL of BASF; polyoxyethylene glyceryl trioleate, for example Tagat™ TO of Goldschmidt; lower alkyl esters of fatty acids, for example ethyl butyrate, ethyl caprylate and ethyl oleate; and water.

Gelatin Capsules

Any pharmaceutically acceptable gelatin capsules can be used to prepare a dosage form of the present invention, including hard and soft gelatin capsules. Such capsules can be prepared according to any suitable process.

i. Hard gelatin capsules

Non-limiting methods for preparing hard gelatin capsules are described in the following patents and/or publications, each of which is hereby incorporated by reference herein.

U.S. Pat. No. 3,656,997 to Cordes.

U.S. Pat. No. 4,231,211 to Strampfer et al.

U.S. Pat. No. 4,263,251 to Voegle.

U.S. Pat. No. 4,403,461 to Goutard et al.

U.S. Pat. No. 4,705,658 to Lukas.

U.S. Pat. No. 4,720,924 to Hradecky et al.

U.S. Pat. No. 4,756,902 to Harvey et al.

U.S. Pat. No. 4,884,602 to Yamamoto et al.

U.S. Pat. No. 4,892,766 to Jones.

U.S. Pat. No. 6,350,468 to Sanso.

International Patent Publication No. WO 84/00919 to Mackie.

International Patent Publication No. WO 85/04100 to Kalidindi.

ii. Soft Gelatin Capsules

In a preferred embodiment, capsule shells are soft gelatin capsule shells. Such shells can be prepared according to any suitable process including but not limited to the plate process, vacuum process, or the rotary die process. See, for example, (1) Ansel et al. (1995) in Pharmaceutical Dosage Forms and Drug Delivery Systems, 6th ed., Williams & Wilkins, Baltimore, Md., pp. 176-182; and (2) Remington: The Science and Practice of Pharmacy, 19th Ed., Mack Publishing Co. Easton. Pa., pp. 1646-1647, the above-recited pages of which are hereby incorporated by reference herein.

Non-limiting examples of suitable methods for preparing soft gelatin capsules are described in the following patents and publications, each of which is hereby incorporated by reference herein.

U.S. Pat. No. 3,592,945 to Pesch.

U.S. Pat. No. 4,609,403 to Wittwer et al.

U.S. Pat. No. 4,744,988 to Brox.

U.S. Pat. No. 4,804,542 to Fischer et al.

U.S. Pat. No. 5,146,758 to Herman.

U.S. Pat. No. 5,254,294 to Wunderlich et al.

U.S. Pat. No. 6,260,332 to Takayanagi.

U.S. Pat. No. 6,238,616 to Ishikawa et al. and

International Patent Publication No. WO 92/15828 to Herman.

As used herein, unless specific context instructs otherwise, the term “capsule shell” (and “gelatin capsule shell”) embraces capsule half-shells (that can cooperate to form a whole capsule shell) and whole capsule shells (that define a fill volume). Such term also embraces soft gelatin capsule shells and hard gelatin capsules, irrespective of the process by which such shells are made.

The terms “sealed capsule shell”, “sealed in a capsule shell”, “sealing in the capsule shell” and the like are meant to denote a whole capsule shell that defines a fill volume, that such fill volume can contain a fill material, that such fill material is enclosed in the whole capsule shell, and that such enclosure affords the fill material more than a de minimus amount of protection from the atmosphere outside of the whole capsule shell.

Utility

Dosage forms of the invention are useful in treatment and prevention of a very wide range of disorders mediated by COX-2, including but not restricted to disorders characterized by inflammation, pain and/or fever. Such dosage forms are especially useful as anti-inflammatory agents, such as in treatment of arthritis, with the additional benefit of having significantly less harmful side effects than compositions of conventional NSAIDs that lack selectivity for COX-2 over COX-1. In particular, dosage forms of the invention have reduced potential for gastrointestinal toxicity and gastrointestinal irritation, including upper gastrointestinal ulceration and bleeding, by comparison with compositions of conventional NSAIDs. Thus dosage forms of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated dosage forms are useful to treat a variety of arthritic disorders, including but not limited to rheumatoid arthritis, spondyloarthropathies, gouty arthritis, osteoarthritis, systemic lupus erythematosus and juvenile arthritis.

Such dosage forms are useful in treatment of asthma, bronchitis, menstrual cramps, preterm labor, tendonitis, bursitis, allergic neuritis, cytomegalovirus infection, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such dosage forms are useful to treat gastrointestinal conditions such as inflammatory bowel disease, Crohn's disease, gastritis, irritable bowel syndrome and ulcerative colitis.

Such dosage forms are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such dosage forms are useful in treatment of ophthalmic disorders, including without limitation inflammatory disorders such as endophthalmitis, episcleritis, retinitis, iriditis, cyclitis, choroiditis, keratitis, conjunctivitis and blepharitis, inflammatory disorders of more than one part of the eye, e.g., retinochoroiditis, iridocyclitis, iridocyclochoroiditis (also known as uveitis), keratoconjunctivitis, blepharoconjunctivitis, etc.; other COX-2 mediated retinopathies including diabetic retinopathy; ocular photophobia; acute trauma of any tissue of the eye including postsurgical trauma, e.g., following cataract or corneal transplant surgery; postsurgical ocular inflammation; intraoperative miosis; corneal graft rejection; ocular, for example retinal, neovascularization including that following injury or infection; macular degeneration; cystoid macular edema; retrolental fibroplasia; neovascular glaucoma; and ocular pain.

Such dosage forms are useful in treatment of pulmonary inflammation, such as that associated with viral infections and cystic fibrosis, and in bone resorption such as that associated with osteoporosis.

Such dosage forms are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such dosage forms are useful in treatment of allergic rhinitis, respiratory distress syndrome, endotoxin shock syndrome and liver disease.

Such dosage forms are useful in treatment of pain, including but not limited to postoperative pain, dental pain, muscular pain, and pain resulting from cancer. For example, such dosage forms are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such dosage forms are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such dosage forms are useful in treatment of angiogenesis-related disorders in a subject, for example to inhibit tumor angiogenesis. Such dosage forms are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

Such dosage forms are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which dosage forms of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such dosage forms can also be used to treat fibrosis that occurs with radiation therapy. Such dosage forms can be used to treat subjects having adenomatous polyps, including those with familial adenomatous polyposis (FAP). Additionally, such dosage forms can be used to prevent polyps from forming in subjects at risk of FAP.

Such dosage forms inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for dosage forms of the invention are for treatment of rheumatoid arthritis and osteoarthritis, for pain management generally (particularly post-oral surgery pain, post-general surgery pain, post-orthopedic surgery pain, and acute flares of osteoarthritis), for treatment of Alzheimer's disease, and for colon cancer chemoprevention.

Besides being useful for human treatment, dosage forms of the invention are useful for veterinary treatment of companion animals, exotic animals, farm animals, and the like, particularly mammals. More particularly, dosage forms of the invention are useful for treatment of COX-2 mediated disorders in horses, dogs and cats.

### EXAMPLES

The following non-limiting examples are provided for illustrative purposes only and are not to be construed as limitations.

### Example 1

Two fill formulations, F1-F2, were prepared as shown in Table 1. One ml of each fill formulation was filled into each of several standard (no sulfite compound present in the shell) soft gelatin capsules (R. P. Scherer).

TABLE 1
Composition of fill formulations F1-F2

| Component | F1 | F2 |
|---|---|---|
| Celecoxib | 278 | 270 |
| PEG400 | 337 | 334 |
| Tween80 | 195 | 195 |
| Oleic Acid | 80 | 78 |
| PVP | — | — |
| Ethanol | — | — |
| Hydroxypropyl methylcellulose (“HPMC”) | 74 | 74 |
| Water | — | 7 |
| Propyl gallate | 2 | 2 |
| Sodium metabisulfite | — | 4 |
| Dimethylamino-ethanol (“DMAE”) | 34 | 35 |
| Total | 34 | 122 |

Filled capsules were placed in a sealed container and store at 40° C. and 75% relative humidity for a period of up to 24 weeks. At various times during storage, capsules were removed from the closed container and evaluated, by visual inspection, for presence or absence of pellicle formation (i.e. cross-linking). Each evaluated capsule was assigned a numerical indicator based on any pellicle observed according to the following scale: (1)=no pellicle; (2)=thin, incomplete pellicle; (3)=thin, complete pellicle; (4)=strong, complete pellicle which inhibits compression of capsule; and (5) thick, strong, and severe pellicle. Pellicle formation observations are shown in Table 2.

TABLE 2
Pellicle formation after storage for up to 8 weeks at 40° C. and
75% relative humidity

| Time | | |
|---|---|---|
| (weeks) | F1 | F2 |
| 0 | 1 | 1 |
| 2 | 3 | 1 |
| 4 | 3 | 1 |
| 6 | 3 | 1 |
| 8 | 4 | 1 |

As shown in Table 2, capsules containing fill formulation F1 (comprising Sodium metabisulfite in an amount of about 3% by weight of the fill material) exhibited no pellicle formation during storage for a period of six months. By contrast, capsules containing fill formulation F2 (no sulfite compound) exhibited pellicle formation by two weeks of storage.

### Example 2

A test material comprising PEG 400 and 414 μtg/ml formaldehyde was prepared. Five aliquots, A1-A4, of the test material were drawn and placed in separate vials. Individually, one of glycine, sodium metabisulfite, sodium bisulfite, or no additional component, was added to each vial in an amount of 5 mg/ml, as shown in Table 3, to form test samples A1-A4, respectively.

TABLE 3
Composition of Test Samples A1-A4

Test Sample

| | A1 | A2 | A3 | A4 |
|---|---|---|---|---|
| Aliquot | A1 | A2 | A3 | A4 |
| Additional | None | Sodium | Sodium | Glycine |
| component | | metabisulfite | bisulfite | |

Each of the test samples were stored at room temperature for a period of three days. After three days of storage, formaldehyde concentration in each sample was measured using HPLC. Amount of formaldehyde present in each sample (% weight of original amount) is shown in Table 4.

TABLE 4
Amount of formaldehyde present in Test Samples A1-A4 after
storage

Test Sample

| | A1 | A2 | A3 | A4 |
|---|---|---|---|---|
| Formaldehyde | 100 | 3.9 | 3.8 | 61.9 |
| content | | | | |

These data show that the sulfite compounds sodium metabisulfite and sodium bisulfite reduced formaldehyde levels upon storage to a greater extent than did glycine. Without being bound by theory, formaldehyde is believed to be a chemical which causes and/or promotes gelatin cross-linking.

### Example 3

The cross-linking behavior of two soft gelatin formulations was investigated over a 6 month period. As shown below (Table 5), Formulation 30 (the control lot) contains dimethylaminoethanol (“DMAE”) and no sulfite. Formulation 19 (the test lot) was similar to the Formulation 30, except that Formulation 19 additionally comprises sodium metabisulfite in the fill material.

TABLE 5
Fill material of Formulations 30 and 19 (mg/g)

| Component | Formulation 30 | Formulation 19 |
|---|---|---|
| celecoxib | 278 | 270 |
| PEG 400 | 337 | 335 |
| Tween 80 | 195 | 195 |
| oleic acid | 80 | 78 |
| HPMC | 74 | 74 |
| DMAE | 34 | 35 |
| propyl gallate | 2 | 2 |
| water | — | 7 |
| sodium metabisulfite | — | 4 |

Both soft gelatin capsule formulations were placed into non-induction-sealed hydroxypropyl ethylene bottles and stored at either 25° C. and 60% RH or 40° C. and 75% RH. Periodically, capsules were tested for degree of cross-linking of the gelatin of the soft gelatin samples as estimated by the drug release profile.

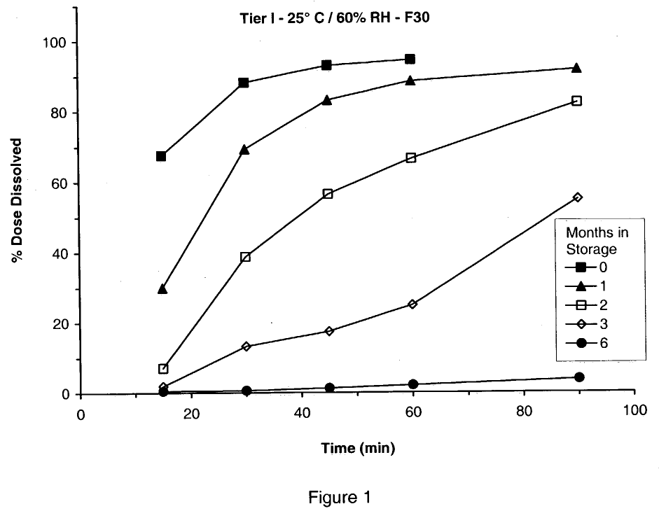

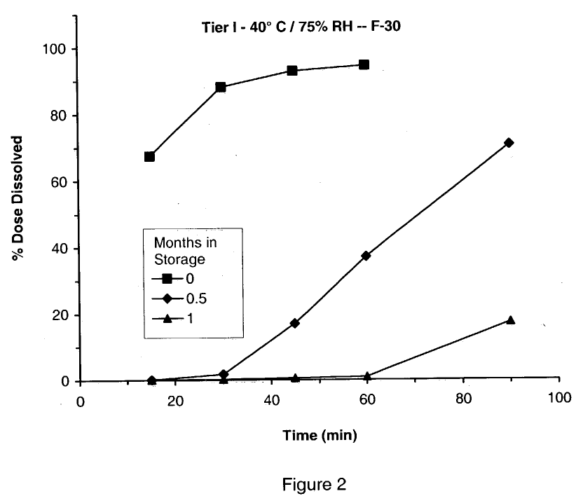

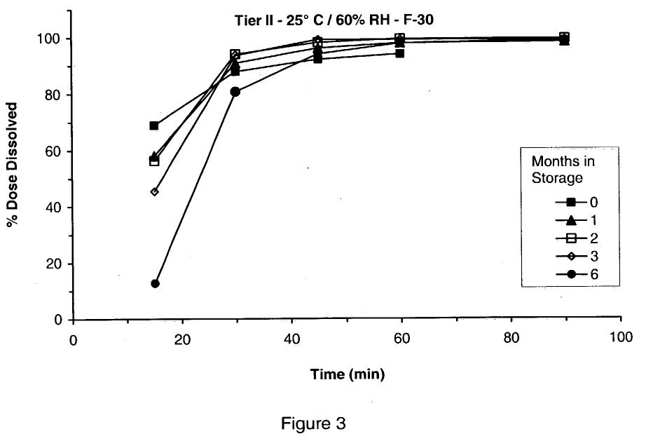

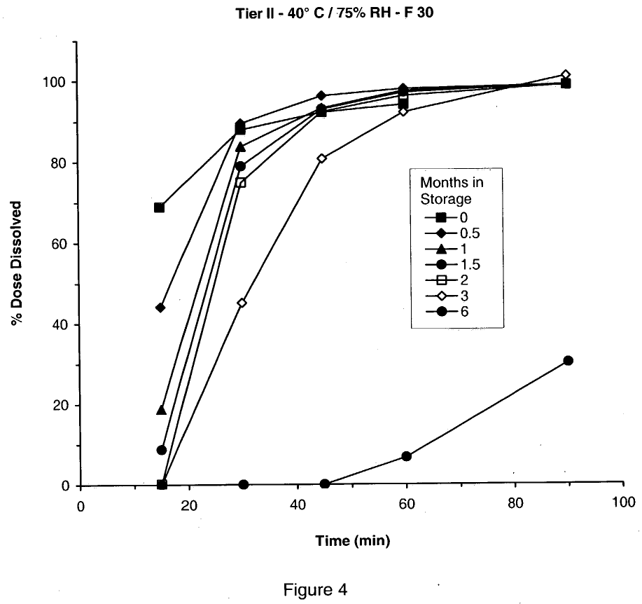

Formulation 30. The Tier I drug release results for control Formulation 30 at 25° C./60% relative humidity (“RH”) and 40° C./75% RH are shown in FIGS. 1 and 2 and the Tier II drug release results for the same lot and conditions are shown in FIGS. 3 and 4. As early as 1 month of storage, there was a marked delay in the Tier I drug release profile at both temperature conditions. This delay increased with storage time. The Tier II drug release profile at 25° C./60% RH and at 40° C./75% RH shows a significant but markedly reduced delay in release profile.

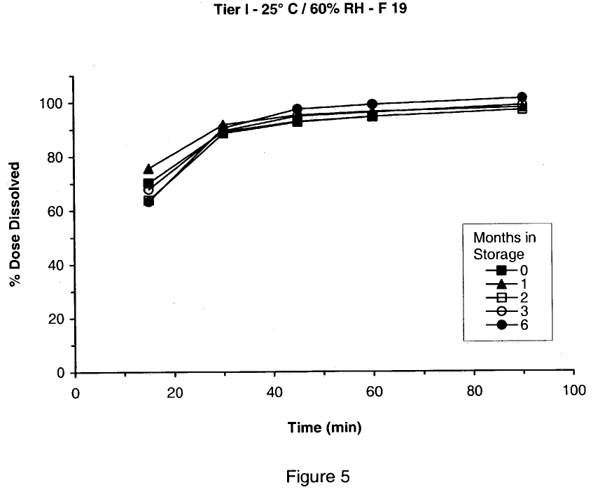

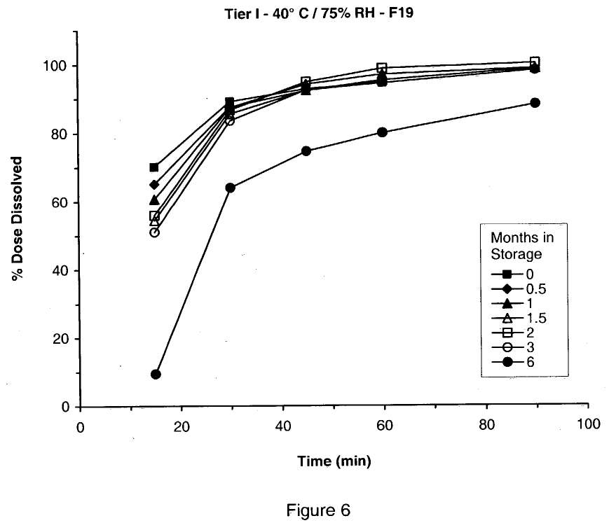

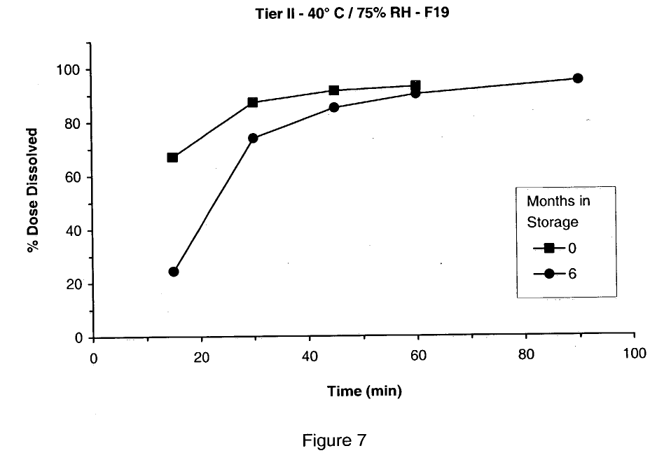

Formulation 19. The Formulation 19 Tier I drug release results for the 25° C./60% RH condition are shown in FIG. 5. No change in the drug release profile is observed through 6 months, indicating that no cross-linking has occurred. Accordingly, the analogous Tier II test for this sample was not performed. FIG. 6 displays the Tier I results for Formulation 19 at 40° C./75% RH. No change in drug release profile is observed for most of the stability time points with the exception of the 6 month time point. To determine if the change in drug release profile at 6 months is a result of cross-linking, the Tier II test was performed on this sample. The Tier II results are displayed in FIG. 7. The Tier I and Tier II results are very similar for this 6 month sample indicating that the change in drug release profile is not attributable to cross-linking.

These data indicate that there was severe cross-linking observed in the Formulation 30. The change in the Tier II drug release profile (i.e. reduced delay) indicates that Tier I delayed release is the result of cross-linking for this formulation and further indicates that a significant delay in the drug release profile in humans would be likely. The Formulation 19, containing sodium metabisulfite, exhibits no measurable gelatin cross-linking through 6 months at stringent (40° C./75% RH) storage conditions. These data demonstrate that the addition of sodium metabisulfite to this formulation significantly reduces the rate of cross-linking and indeed may inhibit cross-linking completely. Without being bound by theory, sodium metabisulfite is believed to inhibit cross-linking by a process in which sodium metabisulfite reacts with aldehydes forming a bisulfite addition product. Thus, sodium metabisulfite may effectively scavenges aldehydes making them unavailable to promote cross-linking in the gelatin.

### Example 4

Four soft gelatin Celecoxib formulations were prepared as shown in Table 6 and tested for pellicle formation at 40° C. and 75% relative humidity (“RH”).

In absence of sulfite, complete pellicle formation was apparent after only 2 weeks storage at 40° C./75% RH (Formulation 30; cross-linking rating=3).

At a Tris concentration of 5 mg/g in the formulation (Formulation 20), delayed pellicle formation but was insufficient to prevent a complete pellicle formation (the cross-linking rating=3) upon 1.5 months storage under 40° C./75% RH.

At a higher Tris concentration in the formulation (26 mg/g, Formulation 50), gelatin cross-linking is completely prevented upon 6 months storage under 40° C./75% RH.

A low sodium metabisulfite (SMB) concentration of 4 mg/g in the formulation (Formulation 19) appeared sufficient to prevent the pellicle formation upon 2 months storage under 40° C./75% RH.

TABLE 6
Gelatin cross-linking analysis of soft gelatin at 40° C./75% RH storage

| Months | | | | | | | | |
|---|---|---|---|---|---|---|---|---|
| at 40° C./ | | | Formulation | | Formulation | | Formulation | |
| 75% | Formulation 50 | | 30 | | 19 | | 20 | |
| RH | mg/ml | | mg/ml | | mg/ml | | mg/ml | |
| | Celecoxib | 200 | Celecoxib | 278 | Celecoxib | 270 | Celecoxib | 270 |
| | PEG400 | 271 | PEG400 | 337 | PEG400 | 335 | PEG400 | 334 |
| | Tween80 | 217 | Tween80 | 195 | Tween80 | 195 | Tween80 | 194 |
| | Oleic acid | 61 | Oleic acid | 80 | Oleic acid | 78 | Oleic acid | 78 |
| | PVP | 47 | | | | | | |
| | EtOH | 113 | | | | | | |
| | HPMC | 38 | HPMC | 74 | HPMC | 74 | HPMC | 74 |
| | | | DMAE | 34 | DMAE | 35 | DMAE | 33 |
| | propyl gallate | 1 | propyl gallate | 2 | propyl gallate | 2 | propyl gallate | 2 |
| | water | 26 | | | water | 7 | water | 10 |
| | Tris | 26 | | | SMB | 4 | Tris | 5 |

| | | | | |
|---|---|---|---|---|
| 0 | 1 | 1 | 1 | 1 |
| 0.5 | | 3 | 1 | 1 |
| 1 | 1 | 3 | 1 | 2 |
| 1.5 | | 3 | 1 | 3 |
| 2 | 1 | 4 | 1 | 3 |
| 3 | 1 | | | |
| 6 | 1 | | | |

### Example 5

In order to gain insight in to the mechanism by which Tris (hydroxymethyl aminomethane) in fill material of a gelatin capsule prevents pellicle formation, a dosage form (of Formulation X-60 set forth in Table 7) was prepared and stored under two different conditions as shown in Table 8. At the times indicated, capsules were removed and Tris content was quantified in the fill material and in the capsule. As shown in Table 8, upon storage with time, Tris content in the capsules increased and Tris content in the fill material decreased in comparison to the initial formulation.

TABLE 7
Soft gelatin capsule Formulation X-60

| Ingredient | Formulation X-60 | |
|---|---|---|
| Celecoxib | 200 | |
| PEG 400 | 271 | |
| Tween 80 | 217 | |
| Oleic acid | 61 | |
| Tris | 26 | |
| Water | 26 | |
| Propyl gallate | 1 | |
| PVP-12PF | 47 | |
| Abs. EtOH | 113 | |
| HPMC-E5 | 38 | |
| Total | 1000 | mg/g |
| Fill Volume | 0.92 | mL |
| (200 mg drug) | | |
| Dosage Form | 18 | Oblong |

soft gelatin capsule

TABLE 8
Tris content in capsule shells following storage of Formulation X-60

| Soft gelatin capsule | Tris in fill | Tris in shell |
|---|---|---|
| Storage conditions | (mg) | (mg) |
| 25° C./60% RH | | |
| T = 2 months | 18.7 | 5.3 |
| T = 6 moths | 17.9 | 6.0 |
| T = 8 months | 16.4 | 6.5 |
| T = 10 months | 17.6 | 7.0 |
| 40° C./75% RH | | |
| T = 2 months | 13.5 | 10.5 |
| T = 6 moths | 10.8 | 11.1 |
| T = 8 months | 10.0 | 10.6 |
| T = 10 months | 10.0 | 13.3 |

26 mg Tris in a soft gelatin capsule at T = 0

What is claimed is:

1. A pharmaceutical dosage form comprising a fill material sealed in capsule shells wherein the fill material comprises (a) a selective cyclooxygenase-2 inhibitory drug of low water solubility and (b) at least one pharmaceutically acceptable sulfite compound, wherein the capsule shells comprise gelatin, and wherein the at least one pharmaceutically acceptable sulfite compound is present in a total sulfite amount sufficient to inhibit gelatin cross-linking and/or pellicle formation in the capsule shells upon storage of the dosage form.

2. The dosage form of claim 1 wherein the at least one pharmaceutically acceptable sulfite compound is selected from the group consisting of sodium metabisulfite, sodium bisulfite, and sodium thiosulfate.

3. The dosage form of claim 1 wherein the at least one pharmaceutically acceptable sulfite compound is present in a total sulfite amount of not more than about 10% of the dosage form on a dry weight basis.

4. The dosage form of claim 1 wherein the fill material further comprises at least one pharmaceutically acceptable excipient selected from the group consisting of free radical-scavenging antioxidants, sweeteners, preservatives, wetting agents, buffering agents, flavoring-agents, colorants, stabilizers, fragrances, glidants, crystallization inhibitors, adhesives, lubricants, and thickeners.

5. The dosage form of claim 1 further comprising at least one free radical-scavenging antioxidant compound selected from the group consisting of α-tocopherols, ascorbic acids, ascorbates, palmitates, butylated hydroxyanisoles, butylated hydroxytoluenes, fumaric acids, fumarates, hypophosphorous acids, malic acids, and alkyl gallates.

6. The dosage form of claim 5 wherein the at least one free radical-scavenging antioxidant is present in a total antioxidant amount of about 0.01% to about 5% of the dosage form on a dry weight basis.

7. The dosage form of claim 1 further comprising at least one sweetener compound selected from the group consisting of mannitols, propylene glycols, sodium saccharins, acesulfame Ks, neotames, and aspartames, sorbitols, sucroses, and high-fructose corn syrups.

8. The dosage form of claim 1 wherein the fill material further comprises at least one preservative compound selected from the group consisting of benzalkonium chlorides, benzethonium chlorides, benzyl alcohols, chlorobutanols, phenols, phenylethyl alcohols, phenylmercuric nitrates, and thimerosals.

9. The dosage form of claim 1 wherein the fill material further comprises at least one surfactant selected from the group consisting of benzalkonium chlorides, benzethonium chlorides, cetylpyridinium chlorides, dioctyl sodium sulfosuccinates, nonoxynol 9, nonoxynol 10, octoxynol 9, poloxamers, polyoxyethylenes (8), caprylic monoglycerides, capric monoglycerides, caprylic diglycerides, capric diglycerides, polyoxyethylene (35) castor oils, polyoxyethylene (20) cetostearyl ethers, polyoxyethylene (40) hydrogenated castor oils, polyoxyethylene (10) oleyl ethers, polyoxyethylene (40) stearates, polysorbate 20s, polysorbate 40s, polysorbate 60s, polysorbate 80s, propylene glycol laurates, sodium lauryl sulfates, sorbitan monolaurates, sorbitan monooleates, sorbitan monopalmitates, sorbitan monostearates, and tyloxapol.

10. The dosage form of claim 1 wherein the fill material is liquid.

11. The dosage form of claim 1 wherein the fill material is self-emulsifying upon contact with gastric fluid.

12. The dosage form of claim 1 wherein the fill material further comprises a solvent.

13. The dosage form of claim 12 wherein the selective cyclooxygenase-2 inhibitory drug and the at least one pharmaceutically acceptable sulfite compound are in solution in the solvent.

14. The dosage form of claim 12 wherein the solvent is present in an amount of about 5% to about 95% of the dosage form on a dry weight basis.

15. The dosage form of claim 12 wherein the solvent comprises at least one of a glycol component and a glycol ether component.

16. The dosage form of claim 12 wherein the solvent comprises a glycol ether component having an average molecular weight of about 75 to about 1000.

17. The dosage form of claim 12 wherein the solvent comprises at least one glycol ether selected from the group consisting of ethylene glycol monomethyl ethers, ethylene glycol dimethyl ethers, ethylene glycol monoethyl ethers, ethylene glycol diethyl ethers, ethylene glycol monobutyl ethers, ethylene glycol dibutyl ethers, ethylene glycol monophenyl ethers, ethylene glycol monobenzyl ethers, ethylene glycol butylphenyl ethers, ethylene glycol terpinyl ethers, diethylene glycol monomethyl ethers, diethylene glycol dimethyl ethers, diethylene glycol monoethyl ethers, diethylene glycol diethyl ethers, diethylene glycol divinyl ethers, ethylene glycol monobutyl ethers, diethylene glycol dibutyl ethers, diethylene glycol monoisobutyl ethers, triethylene glycol dimethyl ethers, triethylene glycol monoethyl ethers, triethylene glycol monobutyl ethers, and tetraethylene glycol dimethyl ethers.

18. The dosage form of claim 12 wherein the solvent comprises at least one glycol selected from the group consisting of propylene glycols, 1,3-butanediol and polyethylene glycols.

19. The dosage form of claim 12 wherein the solvent comprises polyethylene glycol having an average molecular weight of about 100 to about 10,000.

20. The dosage form of claim 12 further comprising at least one co-solvent selected from the group consisting of alcohols, oleic acid triglycerides, linoleic acid triglycerides, caprylic triglycerides, capric triglycerides, caprylic monoglycerides, capric monoglycerides, caprylic diglycerides, capric diglycerides, polyoxyethylene caprylic glycerides, polyoxyethylene capric glycerides, propylene glycol fatty acid esters, polyoxyethylene (35) castor oils, polyoxyethylene glyceryl trioleates, lower alkyl esters of a fatty acid, and water.

21. The dosage form of claim 1 wherein the selective cyclooxygenase-2 inhibitory drug is a compound of formula (I) 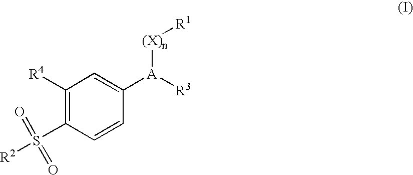
wherein:
A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;
X is O, S or CH₂;
n is 0 or 1;
R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is methyl, amino or aminocarbonylalkyl;
R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and
R⁴ is selected from hydrido and halo.

22. The dosage form of claim 1 wherein the selective cyclooxygenase-2 inhibitory drug is selected from the group consisting of celecoxib, deracoxib, valdecoxib, rofecoxib, etoricoxib, 2-(3,5-difluorophenyl)-3-[4-(methylsulfonyl)phenyl]-2-cyclopenten-1-one, 2-(3,4-difluorophenyl)-4-(3-hydroxy-3-methyl-1-butoxy)-5-[4-(methylsulfonyl)phenyl]-3-(2H)-pyridazinone, and pharmaceutically acceptable salts and prodrugs thereof.

23. The dosage form of claim 1 wherein the cyclooxygenase-2 inhibitory drug is celecoxib.

24. The dosage form of claim 23 wherein the celecoxib is present in an amount of about 10 to about 400 mg.

25. The dosage form of claim 1 wherein the capsule shells are hard gelatin capsule shells.

26. The dosage form of claim 1 wherein the capsule shells are soft gelatin capsule shells.

27. The dosage form of claim 1 wherein each of the gelatin capsule shells have a fill capacity of about 0.1 ml to about 2 ml.

28. The dosage form of claim 1 wherein the capsule shells and/or the fill material further comprise at least one substance that promotes cross-linking of gelatin when in contact therewith, said substance being the selective cyclooxygenase-2 inhibitory drug or an excipient substance, and said substance acting independently or in combination with one or more other substances to promote said cross-linking.

29. The dosage form of claim 28 comprising a first said capsule shell and said fill material and a second said capsule shells and said fill material, said first and second capsule shell and fill material being substantially identical; wherein upon
(a) testing the first capsule shell and fill material in a first in vitro dissolution assay;
(b) storing a second capsule shell and fill material in a closed container maintained at 40° C. and 85% relative humidity for a period of four weeks;
and, after said storage,
(c) testing the second capsule shell and fill material in a second in vitro dissolution assay which is identical to the first in vitro dissolution assay;
the amount of the cyclooxygenase-2 inhibitory drug dissolved at 45 minutes in the second dissolution assay is within ±15 percent of the amount of cyclooxygenase-2 inhibitory drug dissolved at 45 minutes in the first dissolution assay; and wherein the first in vitro dissolution assay is conducted within a reasonably short time after formation of the dosage form.

30. The dosage form of claim 1 wherein the at least one pharmaceutically acceptable sulfite compound is sodium metabisulfite and/or sodium bisulfite in a total sulfite amount of about 0.5% to about 5% on a dry weight basis; wherein the fill material further comprises hydroxypropyl methylcellulose and/or polyethylene glycol, wherein the selective cyclooxygenase-2 inhibitory drug is celecoxib present in an amount of about 10 to about 400 mg, and wherein the capsule shells are soft gelatin capsule shells.
